(19) United States
(12) Patent Application Publication
(10) Pub. No.: US 2006/0052776 A1
(43) Pub. Date: Mar. 9, 2006

(54) Electrosurgical probe

(76) Inventors: Kai Desinger, Berlin (DE); Christoph Janott, Berlin (DE); Andre Roggan, Berlin (DE); Winfried Hohenhorst, Essen (DE)

Correspondence Address: BECK AND TYSVER P.L.L.C., 2900 THOMAS AVENUE SOUTH, SUITE 100, MINNEAPOLIS, MN, 55416, US

(21) Appl. No.: 11/216,847

(22) Filed: Aug. 31, 2005

(30) Foreign Application Priority Data
Sep. 1, 2004 (DE) 10 2004 042 998.7

(51) Int. Cl. A61B 18/14

(52) U.S. Cl. 606/50

(57) ABSTRACT

The invention relates to an electrosurgical probe, in particular for electrocoagulation, comprising a handle and a shaft, the shaft being joined to the handle and having at least two electrodes offset from each other in the axial direction of the shaft, with one electrode closer to the handle forming a proximal electrode and one electrode more distant from the handle forming a distal electrode, wherein said electrodes each form an electrically conductive outer surface of the shaft and are electrically insulated from each other by an insulator, the outer diameter of the two electrodes and the outer diameter of the insulator being approximately the same, wherein a bite piece made of a biocompatible and preferably soft material is provided near the handle on the outside of the shaft in the region of the proximal electrode.

The invention relates to an electrosurgical probe, in particular a coagulation electrode for tongue base reduction, comprising a handle and a shaft joined to the handle, said shaft having a single electrode or, in a preferred variant, at least two electrodes offset from each other in the axial direction of the shaft. In the preferred variant with at least two electrodes, one electrode closer to the handle forms a proximal electrode and the other electrode more distant from the handle forms a distal electrode. A third variant has two laterally offset electrodes which may be disposed in the form of strip electrodes on a common shaft, or on a distally branching shaft, or on two separate shafts projecting from the handle. At least one portion of the outer surfaces of the single electrode or electrodes forms an electrically conductive outer surface of the shaft in each case. If there is a plurality of electrodes, the electrically conductive outer surfaces are axially separated from each other by an insulator. The outer diameter of the electrodes and the outer diameter of the insulator are approximately the same.

The invention further relates to a method for treating the tongue base with such an electrosurgical probe.

In the following, the terms “coagulation electrode” and “electrode arrangement” are used synonymously as terms for special forms of the electrosurgical probe as described herein.

Such coagulation electrodes are basically known, and their uses include tissue reduction of the tongue by means of thermal ablation. This is done by inserting at least one active electrode ventrally under the dorsum of the tongue into the tissue. For example, a high-frequency alternating voltage is applied to the two electrodes in contact with the body tissue, thus producing an alternating current in the tongue tissue surrounding the electrodes and causing said tissue to be heated. The high-frequency current and the electrode geometry are selected such that the heating of the tongue tissue caused by the high-frequency current leads to cell death and hence to tissue obliteration. This enables the treatment of obstructive sleep apnea syndrome (OSAS), for example.

In practice, the high-frequency current is delivered to the tongue tissue using either monopolar electrode arrangements comprising a single active electrode in the shaft, or bipolar arrangements of electrodes comprising two active electrodes in the shaft. In the case of monopolar arrangements, one electrode that is not part of the probe—also referred to as a neutral electrode—is placed in the form of a electrode of large area on the patient's skin near the site of treatment, fixated there and connected to one pole of the alternating voltage source. A second electrode on a probe and handled by the operator—also referred to as an active electrode—is connected to the other pole of the alternating voltage source. The specific form of electrode is adapted to the respective application, in particular to the size of the tongue tissue area being treated, in such a way that both the duration of the operation and the thermal stress imposed on the affected part of the body are within acceptable limits, and that coagulation occurs only in the desired area of tongue tissue.

Other coagulation electrodes for treating tongue tissue with electrode arrangements and high-frequency thermotherapy are known from U.S. Pat. No. 5,707,349, U.S. Pat. No. 6,152,143 and U.S. Pat. No. 6,179,803.

In practice, the disadvantage of these known coagulation electrodes for reducing tongue tissue volume is that their effect on tissue is imprecisely localized. This can lead to errors in treatment or to suboptimal results due to imprecise positioning of the coagulation electrodes.

The object of the invention is therefore to develop an electrosurgical probe, in particular a coagulation electrode of the kind initially specified, such that the operation puts less strain on the patient, on the one hand, and that the operator is able to reduce the volume of tongue tissue in a precisely localized manner, on the other hand.

This object is accomplished according to the invention by an electrosurgical probe of the kind initially specified, in which a bite piece made of a biocompatible and preferably soft material is provided near the handle on the outside of the shaft in the region of the single or the proximal electrode, or the laterally offset electrodes.

A probe of this kind advantageously enables a patient to hold the probe in place with his teeth during treatment. This enables slippage of the electrode arrangement inside the tissue during application of energy to be easily prevented.

In a preferred variant of the invention, the single or the proximal electrode or the laterally offset electrodes are covered on the outside by an insulating layer at the proximal end adjacent the handle.

A suitable design for a bipolar variant of the probe with two electrodes includes an insulator for separating the two electrodes from each other in the longitudinal direction of the shaft.

It is advantageous within the meaning of the invention if the bite piece covers the single or the proximal electrode or the laterally offset electrodes at least partially and preferably encloses a longitudinal portion of the outer surface of the respective electrode completely. The outer diameter of the bite piece is preferably at least twice as great as the outer diameter of the electrode or electrodes and the insulator, in order to allow the probe to be securely held by the patient's teeth. Accordingly, the strength of the soft and biocompatible material of the bite piece in the radial direction relative to the shaft is preferably greater by several factors than the material strength of the insulation layer.

In one preferred variant of the probe, a distal end portion of the single electrode or the distal electrode or the laterally offset electrodes is covered on the outside by an insulating layer. By means of such an insulating layer, it is possible to prevent coagulation on the tongue surface of a tongue base undergoing treatment. This also reduces the risk of pain, infections and ulcerations.

Different alternative configurations of the distal end of a shaft are envisaged, and are characterized in that
the distal end of the shaft is cone-shaped and the distal end of the shaft is pointed, or
the distal end of the shaft is cone-shaped, and the distal end of the shaft is rounded, or
the tip of the shaft is trocar-shaped at the distal end, or
the tip of the shaft is hemispherical at the distal end, or
the tip of the shaft is wedge-shaped at the distal end.

Depending on the type of handling required, the probe may be configured in such a way that the handle and the respective shaft are unbent relative to each other, or that at least one portion of a shaft is bent in the longitudinal direction at an angle of little more than 0° to 90° relative to the handle.

It is preferable that the single electrode or the distal or proximal electrode or both electrodes or the laterally offset electrodes have markings that provide the operator with information about the depths of insertion in the tongue base.

In order to increase the stability of the probe, it is advantageous if a reinforcing pipe is mounted onto the outside of the shaft above the single or the proximal electrode or the laterally offset electrodes in the region of their respective proximal ends, said reinforcing pipe at least partially enclosing the outer surface of the proximal electrode and reinforcing the shaft.

Embodiments of the invention shall now be explained in greater detail with reference to the Figures.

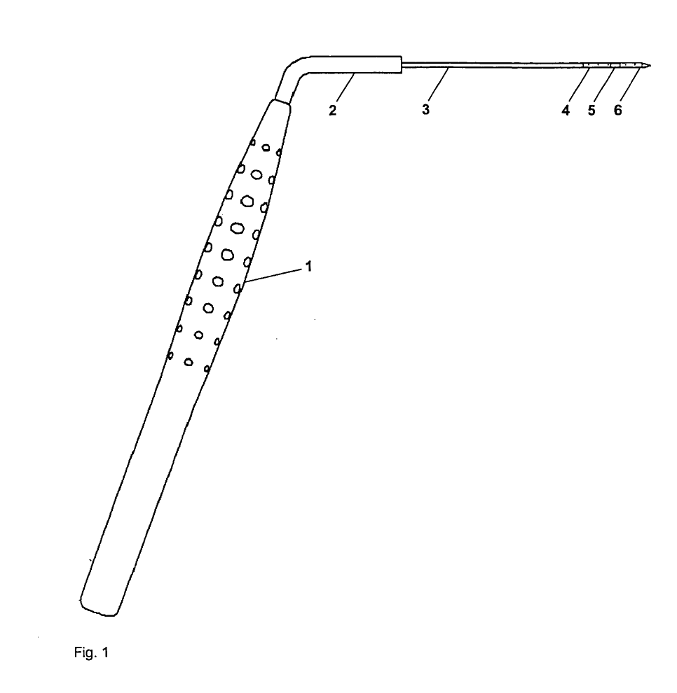

FIG. 1 shows a sample view of an electrode arrangement for a coagulation electrode according to the invention

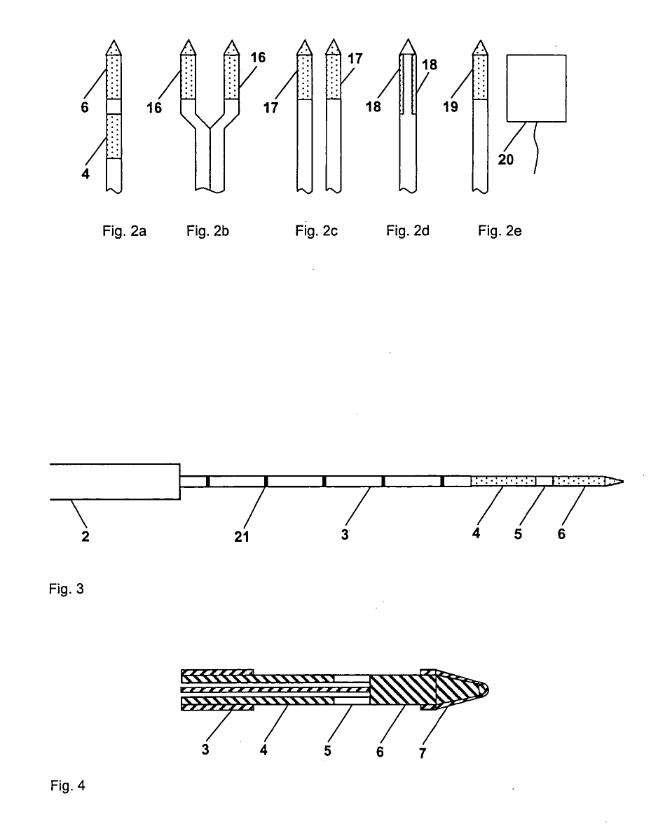

FIGS. 2a-2e show a schematic view of the electrode arrangements for preferred variants of the electrosurgical probe

FIG. 3 shows a partial view of a preferred embodiment of the electrosurgical probe in FIG. 1

FIG. 4 shows a cross-section through another preferred embodiment of the electrode arrangement in FIG. 1

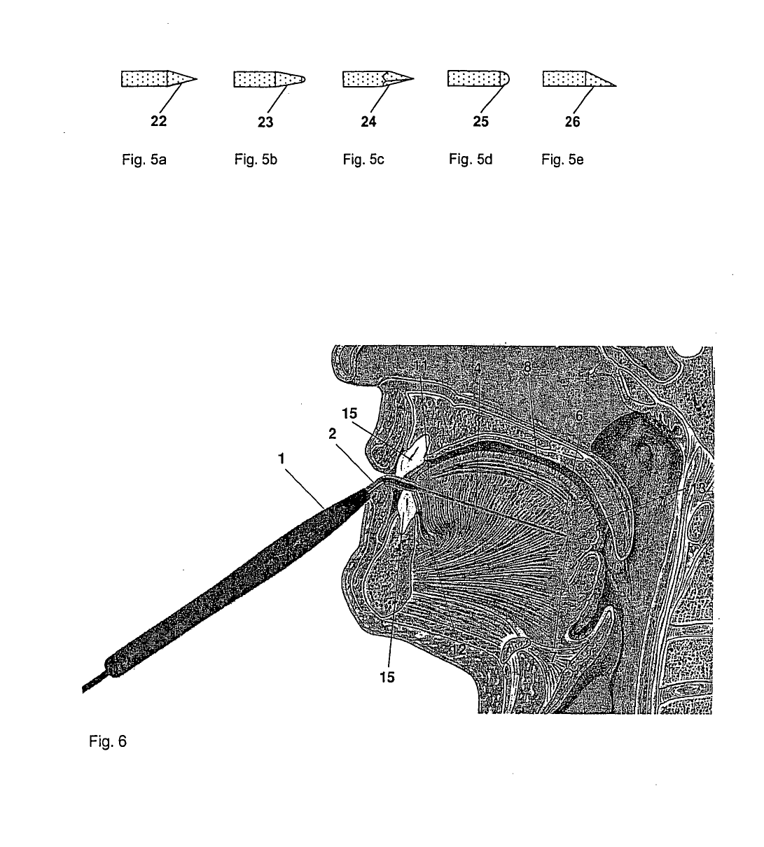

FIG. 5 shows different alternative configurations of the distal end of a corresponding shaft for the electrosurgical probe shown in FIG. 1

FIG. 6 shows a sample schematic view showing use of the invention shown in FIG. 1.

FIG. 1 shows a bipolar coagulation electrode comprising a handle 1 and a shaft joined to the handle and comprising at least two axially offset electrodes. One electrode closer to the handle forms a proximal electrode 4, and the other electrode more distant from the handle forms a distal electrode 6. Said electrodes each form an electrically conductive outer surface of the shaft and are axially separated from each other by an insulator 5. The outer diameter of said electrodes and the outer diameter of the insulator are approximately the same. The outside of proximal electrode 4 is preferably covered at its proximal end with an insulating layer 3. The proximal electrode has a bite piece 2 near the handle, said bite piece preferably consisting of a soft and biocompatible material and being partially applied over the proximal electrode 4 and enclosing the outer surface of the proximal electrode. The material strength of bite piece 2 is preferably greater by several factors than the material strength of insulating layer 3.

Insulating layer 3 is preferably only a few microns thick, for example 1 to 10 μm. Insulating layer 3 is preferably applied to the proximal end of the proximal electrode 4.

The electrode arrangement is preferably rigid and straight in design, the proximal and the distal electrode coaxially aligned so that they can be inserted into the tongue base by means of a straight-line translation movement. The electrode arrangement can thus be advanced so far into the tongue base until the distal end of the distal electrode is touching the tongue surface of the tongue base from the inside. Depending on the intended application, it may also be advantageous for the instrument to be bent in the longitudinal direction.

FIGS. 2a to 2e show electrode arrangements for an electrosurgical probe comprising a handle and a shaft joined to the handle, said shaft having a single active electrode 19 with a neutral electrode 20 (FIG. 2e) or, in a second variant, at least two electrodes axially offset from each other in the direction of the shaft, namely an active proximal electrode 4 and an active distal electrode 6 (FIG. 2a). A third variant has two laterally offset electrodes that may be arranged in the form of strip electrodes 18 on a common shaft (FIG. 2d). A fourth variant has two laterally offset electrodes 16 on a distally branching shaft (FIG. 2b). A fifth variant has two laterally offset electrodes 17 on two separate shafts projecting from the handle (FIG. 2c).

FIG. 3 shows an alternative embodiment in which there are markings 21 on the shaft which provide the operator with information about the depth to which the distal electrode 6 and the proximal electrode 4 are inserted into the tongue base.

In an alternative embodiment, part of which is shown in FIG. 4, an insulating layer 7 is applied to the distal end of distal electrode 6.

Different alternative configurations of the distal end of a shaft are envisaged (FIGS. 5a to 5e), and are characterized in that
the distal end 22 of the shaft is cone-shaped, and the distal end of the shaft is pointed (FIG. 2a), or
the distal end 23 of the shaft is cone-shaped and the distal end of the shaft is rounded (FIG. 5b), or
the tip of the shaft is trocar-shaped 24 at the distal end (FIG. 5c), or
the tip of the shaft is hemispherical 25 at the distal end (FIG. 5d), or
the tip of the shaft is wedge-shaped 26 at the distal end (FIG. 5e).

The advantages of the invention consist, in particular, in the proximal electrode 4 having a bite piece 2 near the handle, said bite piece preferably consisting of a soft and biocompatible material that is partially applied over proximal electrode 4 and enclosing the outer surface of proximal electrode 4. The patient can hold said bite piece 2 with his teeth during treatment. This prevents slippage of the electrode arrangement inside the tissue during the application of energy.

Another advantage of the invention is effected by an alternative embodiment in which an insulating layer 7 is provided on the outside of the distal end of distal electrode 6. Said insulating layer prevents coagulation from occurring on tongue surface 13 of the tongue base. This reduces the risk of pain, infections and ulcerations. The electrode arrangement can thus be advanced so far into the tongue base until the distal end of distal electrode 6 is touching the tongue surface 13 of the tongue base from the inside. The tongue surface 13 forms a natural stop member due to the stability of its tissue structure (multilayered squamous epithelium). This makes it easier for the operator to position the electrodes.

As already mentioned, the electrosurgical probe is used to reduce the volume of tissue in the tongue 8 by means of thermal ablation. This is achieved by using the coagulation electrode described above in accordance with the method described below (see FIG. 6).

In the case of a monopolar probe with only a single active (electrically connected) electrode, a neutral electrode of relatively large area and initially independent of the probe is conductively attached to the patient's body. This step is obviated in the case of a bipolar or multipolar probe.

The shaft with the single electrode 19 (monopolar probe) or with the proximal electrode 4 and distal electrode 6 (axial bipolar probe) or the shaft with the laterally offset electrodes (lateral bipolar probe with strip electrodes 18) or the shafts with electrodes 16, 17 (laterally bipolar, double-shafted probe) is then inserted into the tissue either from the tip of the tongue 11, ventrally under the dorsum of the tongue 12, or dorsally above the tongue 8. The electrode arrangement is advanced into the tongue base until the distal end of the single or distal electrode 6 is touching the tongue surface 13 of the tongue base 13 from the inside, The tongue surface 13 forms a natural stop member due to the stability of its tissue structure (multilayered squamous epithelium). The electrode arrangement is then retracted a little to avoid any coagulation on the tongue surface. The electrode arrangement positioned in this way is fixated by bite piece 2, which the patient holds in place with his teeth 15, thus preventing any slippage. A high-frequency alternating voltage, for example, is then applied to the two electrodes (or to the neutral and probe electrode in the case of a monopolar arrangement), thus producing an alternating current in the tongue tissue surrounding the electrodes and causing said tissue to be heated. The high-frequency current and the electrode geometry are selected such that the heating of the tongue tissue caused by the high-frequency current will lead to cell death and hence to tissue obliteration. The electrode arrangement is then removed again from tongue 8.

In an alternative variant of the method, the shaft with the single electrode 19 (monopolar probe) or with the proximal electrode 4 and distal electrode 6 (axial bipolar probe) or the shaft with the laterally offset electrodes (lateral bipolar probe with strip electrodes 18) or the shafts with electrodes 16, 17 (laterally bipolar, double-shafted probe) is then inserted into the tissue either from the tip of the tongue 11, ventrally under the dorsum of the tongue 12, or dorsally above the tongue 8. The electrode arrangement is advanced into the tongue base until the distal end of the single or distal electrode 6 is touching the tongue surface 13 of the tongue base 13 from the inside, The tongue surface 13 forms a natural stop member due to the stability of its tissue structure (multilayered squamous epithelium). This makes it easier for the operator to position the electrodes. The electrode arrangement positioned in this way is fixated by bite piece 2, which the patient holds in place with his teeth 15, thus preventing any slippage. A high-frequency alternating voltage, for example, is then applied to the two electrodes (or, again, by using the neutral electrode in the case of a monopolar arrangement), thus producing an alternating current in the tongue tissue surrounding the electrodes and causing said tissue to be heated. The high-frequency current and the electrode geometry are selected such that the heating of the tongue tissue caused by the high-frequency current leads to cell death and hence to tissue obliteration. An insulating layer 7 applied on the outside at the distal end of the single or distal electrode 6 prevents coagulation from occurring on tongue surface 13 of the tongue base and reduces the risk of pain, infections and ulcerations. The electrode arrangement is then removed again from tongue 8.

1. Electrosurgical probe, in particular for electrocoagulation, comprising
a handle and
at least one shaft
joined to said handle and
having at least one electrode
which forms an electrically conductive outer surface,
characterized in that
a bite piece made of a biocompatible and preferably soft material is provided on the outside of the shaft near the handle and that the shaft has at least two electrodes offset from each other,
said electrodes forming an electrically conductive outer surface of said shaft and
being electrically insulated from each other by an insulator, and
said bite piece being disposed in the region of the proximal electrode near the handle.

2. Electrosurgical probe according to claim 1,
characterized in that
the electrodes are offset from each other in the axial direction of the shaft, with one electrode closer to the handle forming a proximal electrode and one electrode more distant from the handle forming a distal electrode, and
the outer diameter of the two electrodes and the outer diameter of the insulator are approximately the same, and
said bite piece being disposed in the region of the proximal electrode near the handle.

3. Electrosurgical probe according to claim 2, characterized in that the proximal electrode is covered on the outside by an insulating layer at its proximal end adjacent the handle.

4. Electrosurgical probe according to claim 2, characterized in that the two electrodes are separated from each other by the insulator in the longitudinal direction of the shaft.

5. Electrosurgical probe according to claim 1, characterized in that the bite piece partially covers the proximal electrode.

6. Electrosurgical probe according to claim 1, characterized in that the probe has at least two electrodes offset from each other in the lateral direction,
said electrodes being disposed as strip electrodes on a shaft or on two separate and parallel shafts,
said shaft or shafts each forming an electrically conductive outer surface, and
being electrically insulated from each other by an insulator,
wherein at least one bite piece is disposed on at least one shaft near the handle.

7. Electrosurgical probe according to claim 1, characterized in that the bite piece encloses a longitudinal portion of the outer surface of the electrode.

8. Electrosurgical probe according to claim 1, characterized in that the bite piece has an outer diameter at least twice as great as the outer diameter of the electrode or electrodes.

9. Electrosurgical probe according to claim 3, characterized in that the strength of the soft and biocompatible material of the bite piece in the radial direction relative to the shaft is greater by several factors than the material strength of the insulation layer.

10. Electrosurgical probe according to claim 1, characterized in that a distal end portion of the single electrode or the distal electrode or the laterally offset electrodes is covered on the outside by an insulating layer.

11. Electrosurgical probe according to claim 1, characterized in that the respective distal end of the shaft or shafts is shaped like a cone.

12. Electrosurgical probe according to claim 11, characterized in that the distal end of the shaft or shafts is pointed.

13. Electrosurgical probe according to claim 11, characterized in that the distal end of the shaft or shafts is rounded.

14. Electrosurgical probe according to claim 1, characterized in that the respective tip of the tip of the shaft or shafts is trocar-shaped at the distal end.

15. Electrosurgical probe according to claim 1, characterized in that the respective tip of the tip of the shaft is hemispherical at the distal end.

16. Electrosurgical probe according to claim 1, characterized in that the respective tip of the tip of the shaft or shafts is wedge-shaped at the distal end.

17. Electrosurgical probe according to claim 1, characterized in that at least one portion of the shaft or of at least one shaft is bent in the longitudinal direction at an angle of 0° to 90° relative to the handle.

18. Electrosurgical probe according to claim 1, characterized in that there are markings along the shaft or shafts in the longitudinal direction to provide the operator with information about the depths of insertion in the tongue base, said markings preferably being spaced apart at equal distances to each other.

19. Electrosurgical probe according to claim 1, characterized in that a reinforcing pipe is mounted onto the outside of the proximal end of the single electrode or the proximal electrode or the laterally offset electrodes in the region of their respective proximal ends, said reinforcing pipe at least partially enclosing the outer surface of the proximal electrode and reinforcing the shaft.

20. A method for treating the tongue base using an electrosurgical probe characterized by the steps:
providing an electrosurgical probe having:
a handle and
at least one shaft
joined to said handle and
having at least one electrode
which forms an electrically conductive outer surface,
characterized in that
a bite piece made of a biocompatible and preferably soft material is provided on the outside of the shaft near the handle and that
the shaft has at least two electrodes offset from each other,
said electrodes forming an electrically conductive outer surface of said shaft and
being electrically insulated from each other by an insulator, and
said bite piece being disposed in the region of the proximal electrode near the handle;
inserting the tip of the probe shaft into tongue tissue,
advancing the shaft until the distal end of the single or distal electrode 6 is touching the tongue surface 13 of the tongue base from the inside,
applying a high-frequency alternating voltage and heating the tongue tissue by means of alternating current such that tissue obliteration occurs, and
removing the probe.

21. Method according to claim 2, characterized in that, after insertion and before application of the high-frequency alternating voltage, the probe shaft is retracted so far that a coagulation effect on the tongue surface is avoided.

22. Method according to claim 2 or 21, characterized in that the tip of the shaft is inserted from the tip of the tongue into the tongue tissue.

23. Method according to claim 2 or 21, characterized in that the tip of the shaft is ventrally inserted under the dorsum of the tongue into the tongue tissue.

24. Method according to claim 2 or 21, characterized in that the tip of the shaft is dorsally inserted above the tongue into the tongue tissue.

25. Method according to one of claims 20 to 24, characterized in that, after insertion and prior to any retraction and before applying the alternating voltage, the probe is secured against slippage by the patient holding the bite piece with his teeth.
